(19) United States
(12) Patent Application Publication
(10) Pub. No.: US 2021/0361978 A1
(43) Pub. Date: Nov. 25, 2021

(54) DERMATOLOGICAL PREPARATIONS FOR MAINTAINING AND/OR RESTORING HEALTHY SKIN MICROBIOTA

(71) Applicant: Yun NV, Aartselaar (BE)

(72) Inventors: Sarah Lebeer, Antwerpen (BE); Ingmar Claes, Antwerpen (BE); Eline Oerlemans, Antwerpen (BE); Marianne Van Den Broek, Antwerpen (BE)

(21) Appl. No.: 17/392,895

(22) Filed: Aug. 3, 2021

Related U.S. Application Data
(63) Continuation of application No. 16/311,321, filed on Dec. 19, 2018.

(30) Foreign Application Priority Data
Jun. 21, 2016 (BE) 2016/5454

(51) Int. Cl. A61Q 19/00; A61K 8/99; A61K 9/00; A61K 35/747

(52) U.S. Cl. CPC A61Q 19/00; A61K 8/99; C12N 1/20; A61K 35/747; A61K 9/0014

(57) ABSTRACT

The present invention is directed to the direct application of beneficial or probiotic bacteria to the skin for maintenance of a healthy skin microbiota and to help restore an unbalanced skin microbiota. The application is based on the use of selected Lactobacillus strains as anti-pathogenic agents, in particular L. plantarum, L. pentosus and/or L. rhamnosus, against common skin pathogens, whereby produced acids such as lactic acid are important antimicrobial factors.

### CROSS REFERENCE TO RELATED APPLICATIONS

This application is a continuation of U.S. patent application Ser. No. 16/322,321, filed Dec. 19, 2018, which is a U.S. National Stage Entry of International Patent Application PCT/EP2017/065006, filed Jun. 20, 2017, which claims priority to Belgian Patent Application 2016/5454, filed Jun. 21, 2016, the contents of all of which are hereby incorporated by reference in their entirety.

### SEQUENCE LISTING

Applicant incorporates by reference a CRF sequence listing having file name “eolf-seq1.txt” with a file size of 6.71 kB, created Jul. 29, 2021.

### FIELD OF THE INVENTION

The present invention is directed to the direct topical application of beneficial or probiotic bacteria to the skin for maintenance of a healthy skin microbiota and to help restore an unbalanced skin microbiota. This restoration of a healthy microbiota falls under the term probiotherapy, defined as the use of beneficial micro-organisms or probiotics to restore a healthy microbiota at a site where microbial dysbiosis occurs. The application is based on the use of selected Lactobacillus strains as anti-pathogenic agents, in particular L. plantarum, L. pentosus and/or L. rhamnosus, against common skin pathogens, whereby produced acids such as lactic acid are important antimicrobial factors.

### BACKGROUND TO THE INVENTION

Hence, it was an object of the present invention to provide a solution for subjects suffering from skin conditions due to an aberrant microbial balance on the skin. Thereto, it was found that the topical use of L. plantarum, L. pentosus and/or L. rhamnosus species on the skin is very effective in restoring and/or maintaining a healthy skin microbiota, and is thus very suitable in relieving skin conditions in subjects in need thereof.

Oral formulations comprising Lactobacillus strains have been used before in the treatment of skin conditions like atopic dermatitis. However, oral administration versus direct topical administration are different administration routes and each have a completely different underlying mechanism. In oral administration, in particular a beneficial effect on the general health via immuno-stimulation is intended, whereas by direct dermatological (skin) administration, competition with ‘unwanted’ microorganisms occurs.

Like the gastrointestinal tract, our skin harbours a unique microbial ecosystem. The type of micro-organisms found on the skin depends on a combination of host factors, environmental factors but also topographical location. The role of this microbiota in skin disorders is still not completely unravelled. However, it seems that, at least, some skin disorders are linked to a disturbed microbiota as antimicrobial treatments can improve clinical symptoms (Grice & Segre 2011). For example, in acne vulgaris a correlation has been found with the presence of Propionibacterium acnes (Beylot et al. 2014). Although acne vulgaris is a multifactorial condition and is, among other factors, influenced by hormonal factors, these P. acnes bacteria seem to induce inflammation resulting in inflamed pimples also called papules or pustels. As P. acnes is also found on a healthy skin not causing acne, this suggests that other factors are involved, tipping the balance of the composition of the skin microbiota towards an overgrowth of this bacteria.

Another example of a skin disorder where the microbiota seems to be important is dandruff (Wang et al. 2015; Sugita et al. 2015; Grice & Segre 2011). In people with dandruff, the fungus Malassezia is often overrepresented. Indications that it is this fungus that is a possible cause of the condition, come from the fact that antimycotic treatment improves the symptoms. In contrast, antibacterial therapies do not improve dandruff. Again, other factors are expected to be involved in this skin disorder but the correlation with Malassezia is intriguing.

Similarly as for dandruff, fungal skin infections with Candida albicans or dermatophytes, like Trichophyton spp., seem to be skin disorders linked to a dysbiosis in the skin microbiota as these species are also present on healthy subjects. In the case of Tinea pedis or ‘athlete's foot’ overgrowth of Trichophyton rubrum or T. mentagrophytes is often observed.

The production of lactic acid in combination with possibly other antimicrobial compounds like bacteriocins seems to give protection against aforementioned infections and dysbiotic conditions and lactic acid seems to be active against bacterial, fungal and even viral pathogens. It is for this reason that lactobacilli are considered to be important in the homeostasis of the dynamical dermatological ecosystem. Potential health promoting mechanisms of lactobacilli are i) to preserve a healthy skin pH (+/−5.5), mainly by production of lactic acid; ii) production of antimicrobial compounds and competitive exclusion of pathogens; iii) modulation of immune response and iv) strengthening of the epithelial barrier.

Hence, it was an object of the present invention to provide a solution for subjects suffering from dermatological conditions due to an aberrant microbial balance of the skin. Thereto, it was found that the topical dermatological use of L. plantarum, L. pentosus and/or L. rhamnosus species is very effective in restoring and/or maintaining a healthy microbiota on the skin, and is thus very suitable in relieving dermatological conditions in subjects in need thereof.

Oral formulations comprising Lactobacillus strains have been used before in the treatment of dermatological disorders. However, oral administration versus direct topical administration are different administration routes and each have a completely different underlying mechanism. In oral administration, in particular a beneficial effect on the general health via immuno-stimulation is intended, whereas by direct administration on the skin, competition with ‘unwanted’ microorganisms occur.

### SUMMARY OF THE INVENTION

In a first aspect, the present invention provides a topical skin composition comprising one or more live Lactobacillus species; wherein at least one of said Lactobacillus species is L. plantarum; more in particular a L. plantarum strain having at least 97% sequence similarity with SEQ ID No 4 in its 16S rRNA gene.

In a further aspect, the present invention provides a live Lactobacillus species for use in restoring and/or maintaining a healthy skin microbiota, by topical route, said Lactobacillus species being L. plantarum; more in particular a L. plantarum strain having at least 97% sequence similarity with SEQ ID No 4 in its 16S rRNA gene.

In yet a further aspect, the present invention provides the use of one or more live Lactobacillus species, in the preparation of a topical skin composition for restoring and/or maintaining a healthy skin microbiota; wherein at least one of said Lactobacillus species is L. plantarum; more in particular a L. plantarum strain having at least 97% sequence similarity with SEQ ID No 4 in its 16S rRNA gene.

The present invention also provides a method for restoring and/or maintaining a healthy skin microbiota; comprising at least one step of administering by topical route, to an individual, an effective amount of one or more live Lactobacillus species; wherein at least one of said Lactobacillus species is L. plantarum; more in particular a L. plantarum strain having at least 97% sequence similarity with SEQ ID No 4 in its 16S rRNA gene.

In yet another aspect, the present invention provides a composition comprising one or more live Lactobacillus species for use in restoring and/or maintaining a healthy skin microbiota, by topical route, said Lactobacillus species being selected from the list comprising L. plantarum, L. pentosus and L. rhamnosus; more in particular a L. plantarum strain having at least 97% sequence similarity with SEQ ID No 4 in its 16S rRNA gene, a L. pentosus strain having at least 97% sequence similarity with SEQ ID No 1 in its 16S rRNA gene and a L. rhamnosus strain having at least 97% sequence similarity with SEQ ID No 5 in its 16S rRNA gene.

The present invention further provides a Lactobacillus strain being L. rhamnosus YUN-S1.0 deposited under accession number LMG P-29611 (deposited at BCCM on May, 12, 2016).

Ina particular aspect, the present invention provides a composition comprising one or more Lactobacillus strains as defined herein above.

In a particular embodiment, the composition of the present invention is a topical skin composition, more in particular in the form of a gel, cream, foam, lotion or ointment.

In another particular embodiment, the present invention provides the Lactobacillus strain as defined herein above or the compositions as defined herein above; for use in restoring and/or maintaining a healthy skin microbiota, by topical route.

In a particular aspect, the present invention provides a topical use of one or more live Lactobacillus species in probiotherapy of the skin; wherein said Lactobacillus species are selected from the list comprising L. plantarum, L. pentosus and L. rhamnosus; more in particular, said probiotherapy consists of restoring and/or maintaining a healthy skin microbiota in a subject in need thereof.

In another particular embodiment, said Lactobacillus species in the topical uses, methods and compositions as disclosed herein, is a Lactobacillus strain selected from the list comprising L. plantarum YUN-V2.0 deposited under accession number LMG P-29456 (deposited at BCCM on Mar. 9, 2016), L. pentosus YUN-V1.0 deposited under accession number LMG P-29455 (deposited at BCCM on Mar. 9, 2016); and L. rhamnosus YUN-S1.0 deposited under accession number LMG P-29611 (deposited at BCCM on May, 12, 2016).

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Characteristics of lactobacilli in reference to growth, production of D- and L-lactic acid (LA) and lowering of the pH of the medium.

FIG. 2: Time course experiment for the analysis of the antipathogenic effect of spent culture supernatant of lactobacilli against Propionibacterium acnes. Growth of the bacteria (optical density at 600 nm; Y-axis) is measured in time (X-axis). Each graph shows replicates of growth of P. acnes. It can be clearly noted that without any addition of antibiotic or SCS, P. acnes quickly starts to grow (NC1). Similar as when erythromycin at 50 μg/ml is added, SCS of all lactobacilli prevents growth of P. acnes while SCS of streptococci or staphylococci does not inhibit growth. *Erythromycine (50 ug/ml); ^{#}Erythromycine (5 ug/ml); ^{§} Minocycline (20 μg/ml) NC1=medium control; NC2=MRS at pH4.3; Numbers 1 to 22=lactobacilli strains (for details see table 1); St=Streptococcus thermophilus; Ss=Streptococcus salivarius; Se=Staphylococcus epidermidis; T0.5=0.5% Tween 80; T1=1% Tween 80.

### DETAILED DESCRIPTION OF THE INVENTION

The present invention is based on the discovery of specific Lactobacillus strains that can compete with growth of Propionibacterium acnes, Candida albicans, Malassezia spp., Trichophyton spp. and bacteria or fungi that are linked with skin conditions like acne vulgaris, dandruff, tinea pedis or other fungal skin infections. These selected strains are herein generally termed “YUN” strains and are capable of competing with skin pathogens and thereby restore a healthy skin microbiota. This restoration of a healthy microbiota falls under the term probiotherapy, defined as the use of beneficial micro-organisms or probiotics to restore a healthy microbiota at a site where microbial dysbiosis occurs.

Hence, in a first aspect, the present invention provides a topical skin composition comprising one or more live Lactobacillus species; wherein at least one of said Lactobacillus species is L. plantarum; more in particular a L. plantarum strain having at least 97% sequence similarity with SEQ ID No 4 in its 16S rRNA gene.

Said composition according to the present invention may comprise further Lactobacillus species such as for example selected from the non-limiting list comprising L. pentosus, L. gasseri, L. crispatus, L. acidophilus, L. jensenii, L. fermentum, L. rhamnosus.

In the context of the present invention, the term “topical” is meant to be the local delivery at a specified location of the body, in particular the application to a particular place on the body. In particular, it includes the application via non-solid formulations such as creams, foams, gels, lotions or ointments. The term “topical” is not meant to include the delivery in the form of solid preparations such as capsules, tablets, . . . .

Hence, the term “topical skin” is meant to include the local delivery using non-solid formulations directly onto the skin of the body. Preferably, the compositions according to the present invention are applied over a large area of the skin in order to be most effective.

In the context of the present invention the term “live Lactobacillus species” is meant to be viable Lactobacillus species, and is not meant to be fragments, culture supernatants, or killed forms thereof.

In a further aspect, the present invention provides a live Lactobacillus species for use in probiotherapy of the skin, by topical route, said Lactobacillus species being L. plantarum; more in particular a L. plantarum strain having at least 97% sequence similarity with SEQ ID No 4 in its 16S rRNA gene. As already defined herein above, said probiotherapy is meant to be the restoration and/or maintenance of a healthy skin microbiota in a subject in need thereof.

Subjects that may benefit from such probiotherapy are for example people/persons with a skin conditions linked to a disturbed skin microbiota possibly due to bacterial or yeast infections and/or any dysbiosis caused by overgrowth of specific pathogenic micro-organisms, like acne vulgaris, tinea pedis, dandruff, rosaceae, impetigo, . . . .

Hence, in a further aspect, the present invention provides the use of one or more live Lactobacillus species, in the preparation of a topical skin composition for restoring and/or maintaining a healthy skin microbiota; wherein at least one of said Lactobacillus species is L. plantarum; more in particular a L. plantarum strain having at least 97% sequence similarity with SEQ ID No 4 in its 16S rRNA gene.

The present invention also provides a method for restoring and/or maintaining a healthy skin microbiota; comprising at least one step of administering by topical route, to an individual, an effective amount of one or more live Lactobacillus species; wherein at least one of said Lactobacillus species is L. plantarum; more in particular a L. plantarum strain having at least 97% sequence similarity with SEQ ID No 4 in its 16S rRNA gene.

In yet another aspect, the present invention provides a composition comprising one or more live Lactobacillus species for use in restoring and/or maintaining a healthy skin microbiota, by topical route, said Lactobacillus species being selected from the list comprising L. plantarum, L. pentosus and L. rhamnosus; more in particular a L. plantarum strain having at least 97% sequence similarity with SEQ ID No 4 in its 16S rRNA gene; a L. pentosus strain having at least 97% sequence similarity with SEQ ID No 1 in its 16S rRNA gene and a L. rhamnosus strain having at least 97% sequence similarity with SEQ ID No 5 in its 16S rRNA gene.

The present invention further provides a Lactobacillus strain selected from the list comprising L. pentosus YUN-V1.0 deposited under accession number LMG P-29455 (deposited at BCCM on Mar. 9, 2016); L. plantarum YUN-V2.0 deposited under accession number LMG P-29456 (deposited at BCCM on Mar. 9, 2016); and L. rhamnosus YUN-S1.0 deposited under accession number LMG P-29611 (deposited at BCCM on May, 12, 2016)

The microbiological deposits mentioned herein, have been made with the BCCM/LMG Bacteria collection (“Belgian co-ordinated collections of micro-organism”) with correspondence address: Laboratorium voor Microbiologie, Universiteit Gent, K. L. Ledeganckstraat 35-9000 Gent, Belgium

Lactobacillus pentosus YUN-V1.0 is a single colony isolate obtained in our lab after subculturing of a strain, that was originally a vaginal isolate of healthy woman. The 16S rRNA gene sequence (SEQ ID No 1) for strain L. pentosus YUN-V1.0 was determined by PCR using primers 8F (5′-AGAGTTTGATCCTGGCTCAG-3′—SEQ ID No 2) and 1525R (5′-AAGGAGGTGATCCAGCCGCA-3′—SEQ ID No 3).

YUN-V2.0 and YUN-V3.0 are single colony isolates obtained in our lab after subculturing of Lactobacillus plantarum strains that were originally isolated from human saliva and a maize silage respectively. The 16S rRNA gene sequence (SEQ ID No 4) for strain L. plantarum YUN-V2.0 was determined by PCR using primers 8F (5′-AGAGTTTGATCCTGGCTCAG-3′—SEQ ID No 2) and 1525R (5′-AAGGAGGTGATCCAGCCGCA-3′—SEQ ID No 3).

YUN-S1.0 is a single colony isolate obtained in our lab after subculturing of a Lactobacillus rhamnosus strain that was originally isolated from a healthy person. The 16S rRNA gene sequence (SEQ ID No 5) for strain L. rhamnosus YUN-S1.0 was determined by PCR using primers 8F (5′-AGAGTTTGATCCTGGCTCAG-3′—SEQ ID No 2) and 1525R (5′-AAGGAGGTGATCCAGCCGCA-3′—SEQ ID No 3).

These particular “YUN” strains can either be used as such, or are preferably formulated in a composition comprising such strains. Said compositions are topical skin compositions more in particular in the form of non-solid formulations such as creams, foams, gels, lotions or ointments.

In particular, the present invention provides the above defined “YUN” strains for use in probiotherapy of the skin, i.e. for restoring and/or maintaining a healthy skin microbiota.

In yet a further aspect, the present invention provides a topical use of one or more live Lactobacillus species in probiotherapy of the skin; wherein said Lactobacillus species are selected from the list comprising L. plantarum, L. pentosus and L. rhamnosus; more in particular, said probiotherapy consists of restoring and/or maintaining a healthy skin microbiota in a subject in need thereof.

In a specific embodiment, the Lactobacillus species in the topical uses, methods and compositions as disclosed herein, is a Lactobacillus strain selected from the list comprising L. plantarum YUN-V2.0 deposited under accession number LMG P-29456 (deposited at BCCM on Mar. 9, 2016); L. pentosus YUN-V1.0 deposited under accession number LMG P-29455 (deposited at BCCM on Mar. 9, 2016); and L. rhamnosus YUN-S1.0 deposited under accession number LMG P-29611 (deposited at BCCM on May, 12, 2016).

### Examples

Materials and Methods

Bacterial Strains and Growth Conditions

Lactobacillus strains (Table 1) were grown at 37° C. in de Man, Rogosa and Sharpe (MRS) medium (Carl Roth). All bacteria were grown in non-shaking conditions and inoculated from glycerol stocks (−80° C.). Solid media contained 1.5% (w/v) agar.

TABLE 1
Bacterial strains used in this research

| | | | | |
|---|---|---|---|---|
| | | | | Reference |
| | | | Relevant genotype | and/or |
| Species | # | Strain | or description | Source |

LACTOBACILLI

| | | | | |
|---|---|---|---|---|
| Lactobacillus | 1 | ATCC- | Single colony isolate obtained | ATCC |
| casei | | 334 | in our lab from a stock | |
| | | | culture of ATCC334 | |
| Lactobacillus | 2 | DN- | Single colony isolate obtained | Commercial |
| casei | | 114001 | in our lab from a | probiotic |
| | | | commercially available | product |
| | | | fermented drink (Actimel ®) | |
| | | | containing L. casei DN-114001, | |
| | | | confirmed by sequencing | |
| Lactobacillus | 3 | Shirota | Single colony isolate obtained | Commercial |
| casei | | | in our lab from a | probiotic |
| | | | commercially available | product |
| | | | fermented drink containing | |
| | | | L. casei Shirota (Yakult ®), | |
| | | | confirmed by sequencing | |
| Lactobacillus | 4 | YUN- | Single colony isolate | |
| pentosus | | V1.0 | | |
| Lactobacillus | 5 | LMG- | Single colony isolate from L. | ATCC |
| plantarum | | 1284 | plantarum ATCC8014 or | |
| | | | LMG1284 | |
| Lactobacillus | 6 | RC-14 | Single colony isolate obtained | Commercial |
| reuteri | | | in our lab from a commercially | probiotic |
| | | | available probiotic supplement | product |
| | | | containing L. reuteri RC-14, | |
| | | | confirmed by sequencing | |
| Lactobacillus | 7 | YUN- | Clinical isolate | |
| rhamnosus | | S1.0 | | |
| Lactobacillus | 12 | GR-1 | Single colony isolate obtained | (Chan et al. |
| rhamnosus | | | in our lab from a commercially | 1984; 1985; |
| | | | available probiotic supplement | Reid 1999; |
| | | | containing L. rhamnosus GR-1 | Reid & |
| | | | | Bruce 2001), |
| | | | | ATCC |
| Lactobacillus | 14 | AMB-2 | single colony isolate | Commercial |
| helveticus | | | | probiotic |
| | | | | product |
| Lactobacillus | 15 | YUN- | Single colony isolate | |
| plantarum | | V2.0 | | |
| Lactobacillus | 16 | 5057 | Single colony isolate | |
| plantarum | | | | |
| Lactobacillus | 17 | LMG- | Single colony isolate obtained | BCCM/ |
| paracasei | | 12586 | in our lab from a stock culture | LMG |
| | | | of LMG12586 | |
| Lactobacillus | 22 | / | Single colony isolate | |
| plantarum | | | | |
| Lactobacillus | 25 | LMG- | Single colony isolate | BCCM/ |
| pentosus | | 8041 | | LMG |

PATHOGENS

| | | | | |
|---|---|---|---|---|
| Trichophyton | 2 | / | Clinical isolate | BCCM/ |
| rubrum | | | | LMG |
| Malassezia | | / | Clinical isolate | BCCM/ |
| furfur | | | | LMG |
| Candida | / | / | Clinical isolate | |
| albicans | | | | |

Preparation of Spent Culture Supernatant (SCS) of Selected Strains

To obtain spent culture supernatant (SCS) containing the secreted active antimicrobial products, growth medium specific for each species was inoculated from a preculture and incubated for 24 h. SCS was obtained by centrifugation for 30 min. at 6797 g (8000 rpm) at 4° C. Afterwards, the SCS was filter sterilized (0.20 μm cellulose acetate, VWR).

Antimicrobial Activity Assays for Co-Cultures of Live Lactobacilli Against Malassezia furfur, Trichophyton Rubrum, Propionibacterium Acnes and Candida Albican.

The antimicrobial activity of the selected bacteria was explored by standard antimicrobial tests with some minor modifications. The antimicrobial activity of the selected bacteria was explored by spot assay (Schillinger and Liicke 1989). Briefly, 1-3 μL of each culture was spotted on an agar plate. These plates were incubated for 24 h up to 72 h depending on the strain. Next, an overnight culture of the pathogen was diluted into 7 mL of soft agar of the medium of the pathogen and poured over the plates with the spots of the selected strains. The plates were incubated overnight at 30-37° C., after which the inhibition zones were measured. A spot of miconazole (for fungi) and/or 0.1% hexetidine and/or tetracycline (for Propionibacterium acnes) was added to the spot plate as positive control before the soft agar was poured.

Radial Diffusion Test of SCS of Lactobacilli

In addition, the antimicrobial activity of spent culture supernatant (SCS) was investigated with a protocol as previously described for the competition assays between lactobacilli and gastro-intestinal pathogens (Coconnier et al. 1997). Miconazol (for fungi) and tetracycline (for Propionibacterium acnes) was used as a positive control. Sterile growth medium was used as a negative control.

Time Course Analysis of the Antimicrobial Activity of SCS of the Selected Strains Against Candida, Propionibacterium Acnes, Malassezia Furfur and Trichophyton Spp (Further Referred to as ‘Pathogens’).

The time course analysis was performed similarly as described previously (De Keersmaecker et al. 2006) with minor modifications. Briefly, an overnight culture of the pathogen was added to the wells of a microplate filled with 50-80% the proper medium supplemented with 50-5% SCS of lactobacilli. MRS at pH 4.3 and antibiotics or antimycotics at the proper concentration were used as a negative and a positive control, respectively. Bacteria or fungi were grown and the optical density (OD) was measured at 590 nm each 30 min during 3 days using a Synergy HTX multi-mode reader (Biotek). Each test was measured at least in triplicate and the average OD was calculated. The antimicrobial activity was expressed as the relative optical density reached after 24 h (stationary phase) compared to the negative controls.

Antibiotic Susceptibility

Antibiotic sensitivity was evaluated using the Kirby-Bauer disc diffusion test. In short, antibiotics were spotted on paper discs and the bacterial inhibition zone was measured on agar plates. The antibiotics tested were erythromycin, normocin, tetracyclin, ampicillin and clindamycin at relevant concentrations.

Proof-of-Concept Human Clinical Trial in Patients with Acne Vulgaris

A proof-of-concept clinical trial was performed on 20 patients with acne vulgaris. Patients were men between 12-25 years with mild inflammatory acne. The aim of this proof-of-concept trial was to assess the impact of a topical probiotic cream (containing +−10-8 colony forming units (CFU) of L. pentosus YUN-V1.0, +−10-8 CFU of L. plantarum YUN-V2.0 and +−10-8 CFU L. rhamnosus YUN-S1.0 per application of 1 g of the topical cream ACN) on the skin microbiota and on the acne severity. Patients were asked to apply the cream twice daily for 56 days (8 weeks). The patients were seen by a dermatologist at start (before the therapy), week 4, week 8 and week 10. A skin swab was taken at each visit. Bacterial DNA was isolated from these samples by the commercial MoBio Powersoil kit (cfr. Human Microbiome Project). Isolated DNA was analysed via 16S rRNA amplicon sequencing with MiSeq Illumina and a bio-informatical analysis was performed. Moreover, a clinical scoring was performed and a photograph taken at each visit.

Proof-of-Concept Human Clinical Trial in Patients with Tinea Pedis (Athlete's Foot)

A proof-of-concept clinical trial was performed on 20 patients with tinea pedis. Patients were between 18-65 years having tinea pedis. The aim of this proof-of-concept trial was to assess the impact of a topical probiotic cream (containing +−10-8 colony forming units (CFU) of L. pentosus YUN-V1.0, +−10-8 CFU of L. plantarum YUN-V2.0 and +−10-8 CFU L. rhamnosus YUN-S1.0 per application of 1 g of of the topical cream FNG) on the skin microbiota and on the Trichophyton infection. Patients were asked to apply the cream twice daily for 56 days (8 weeks). The patients were seen by a dermatologist at start (before the therapy), week 4, week 8 and week 10. A skin swab was taken at each visit. Bacterial DNA was isolated from these samples by the commercial MoBio Powersoil kit (cfr. Human Microbiome Project). Isolated DNA was analysed via 16S rRNA amplicon sequencing with MiSeq Illumina and a bio-informatical analysis was performed. For analysis of the presence of the fungi, swabs were also plated out on Trichophyton specific medium (medium suggested by BCCM). Colony PCR using universal ITS (‘internal transcribed region’) primers ITS1 (SEQ ID No 6) (5′-TCCGTAGGTGAACCTGCGG-3′) and ITS4 (SEQ ID No 7) (5′-TCCTCCGCTTATTGATATGC-3′) followed by sequencing was performed to identify the fungi. Moreover, a clinical scoring was performed and a photograph taken at each visit.

Results

Growth Characteristics and Lactate Production

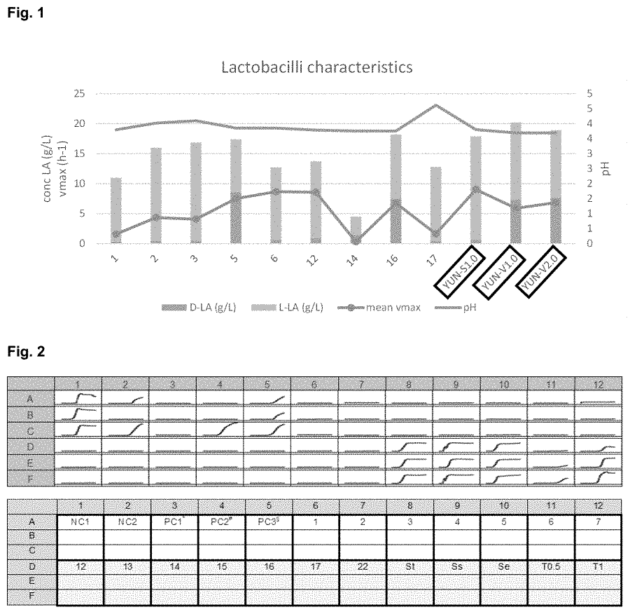

Possible beneficial or probiotic strains were characterized in terms of growth characteristics, lactate production and ability of lowering of the pH of the medium. These characteristics are expected to be important for the antipathogenic activity. These data show that Lactobacillus pentosus YUN-V1.0 and L. plantarum YUN-V2.0 and L. rhamnosus YUN-S1.0 produce the highest amount of lactic acid (FIG. 1).

Antipathogenic Activity Against Propionibacterium acnes

Time course experiments were performed analyzing the antimicrobial activity of spent culture supernatant (SCS) of the selected strains against Propionibacterium acnes. SCS of all tested strains inhibited the growth of Propionibacterium acnes while SCS of other bacterial species like Streptococcus thermophilus and S. salivarius, both also lactic acid bacteria, and Staphylococcus epidermidis did not inhibit growth of P. acnes. This suggests species and perhaps strain specific properties of the selected lactobacilli to be important for the antipathogenic activity against P. acnes (FIG. 2).

Antipathogenic Activity Against Malassezia, Trichophyton and Candida

In a next phase, the beneficial or probiotic bacteria were screened for their antipathogenic effect against specific skin pathogens. The results of a spot assays against Malassezia furfur, Trichophyton rubrum and Candida albicans are shown in table 1, 2 and 3 respectively.

TABLE 1
Spot assay of selected lactobacilli against Malassezia furfur.

| | Malassezia furfur | | |
|---|---|---|---|
| Strain | Exp 1 | Exp 2 | Exp 3 |
| 1 | ++ | − | + |
| 2 | ++ | − | + |
| 3 | + | + | − |
| 4 | ++ | +++ | ++ |
| 5 | +++ | ++ | ++ |
| 6 | + | ++ | ++ |
| 7 | ++ | − | − |
| 12 | + | − | − |
| 13 | + | − | + |
| 14 | − | + | − |
| 15 | +++ | +++ | +++ |
| 16 | ++ | ++ | ++ |
| 17 | − | + | − |
| 22 | +++ | ++ | ++ |
| 25 | ++ | ++ | + |

*three independent repeats are shown

TABLE 2
Spot assay of selected lactobacilli against Trichophyton rubrum.

| | Trichophyton rubrum | | |
|---|---|---|---|
| Strain | Exp 1 | Exp 2 | Exp 3 |
| 1 | + | ++ | +++ |
| 2 | + | ++ | ++ |
| 3 | + | ++ | ++ |
| 4 | ++ | ++ | +++ |
| 5 | ++ | ++ | +++ |
| 6 | − | − | +++ |
| 7 | ++ | +++ | + |
| 12 | ++ | +++ | +++ |
| 13 | ++ | − | − |
| 14 | + | ++ | ++ |
| 15 | +++ | +++ | +++ |
| 16 | ++ | +++ | +++ |
| 17 | + | +++ | ++ |
| 22 | ++ | +++ | +++ |
| 25 | ++ | +++ | +++ |

*three independent repeats are shown

TABLE 3
Radial diffusion assay of selected lactobacilli against Candida albicans.

| | Candida albicans | | |
|---|---|---|---|
| Strain | Exp 1 | Exp 2 | Exp 3 |
| 1 | − | − | − |
| 2 | + | + | + |
| 3 | + | + | + |
| 4 | ++ | ++ | ++ |
| 5 | + | + | + |
| 6 | − | − | − |
| 7 | + | + | ++ |
| 12 | + | + | + |
| 13 | / | / | / |
| 14 | + | − | − |
| 15 | + | + | ++ |
| 16 | + | + | + |
| 17 | − | − | − |
| 22 | / | / | / |
| 25 | / | / | / |

*three independent repeats are shown

Spent culture supernatant from L. pentosus YUN-V1.0 and L. plantarum YUN-V2.0 was also tested in radial diffusion assays and demonstrated to be efficient in inhibiting Malassezia, Trichophyton and Candida growth. L. rhamnosus YUN-S1.0 was not as efficient in inhibiting growth of Malassizia but was able to inhibit growth of Trichophyton and Candida.

Antibiotic Susceptibility

The selected bacteria were also tested for their antibiotic susceptibility as to prevent spreading of antibiotic resistance genes. All lactobacilli were susceptible to erythromycin, normocin, tetracyclin, ampicillin and clindamycin, except for L. plantarum 5057, which was susceptible to tetracyclin. For this reason, strain L. plantarum 5057 was found not to be suitable to use as a strain for probiotherapy.

### REFERENCES

Beylot, C. et al., 2014. Propionibacterium acnes: an update on its role in the pathogenesis of acne. Journal of the European Academy of Dermatology and Venereology: JEADV, 28(3), pp. 271-8.
Chan, R. C. et al., 1985. Competitive exclusion of uropathogens from human uroepithelial cells by Lactobacillus whole cells and cell wall fragments. Infection and immunity, 47(1), pp. 84-9.
Chan, R. C., Bruce, A. W. & Reid, G., 1984. Adherence of cervical, vaginal and distal urethral normal microbial flora to human uroepithelial cells and the inhibition of adherence of gram-negative uropathogens by competitive exclusion. The Journal of urology, 131(3), pp. 596-601.
Grice, E. A. & Segre, J. A., 2011. The skin microbiome. Nature reviews. Microbiology, 9(4), pp. 244-53.
Reid, G., 1999. The Scientific Basis for Probiotic Strains of Lactobacillus. Appl. Envir. Microbiol., 65(9), pp. 3763-3766.
Reid, G. & Bruce, A. W., 2001. Selection of lactobacillus strains for urogenital probiotic applications. The Journal of infectious diseases, 183 Suppl, pp. S77-80.
Sugita, T. et al., 2015. Temporal changes in the skin Malassezia microbiota of members of the Japanese Antarctic Research Expedition (JARE): A case study in Antarctica as a pseudo-space environment. Medical mycology, 53(7), pp. 717-24.
Wang, L. et al., 2015. Characterization of the major bacterial-fungal populations colonizing dandruff scalps in Shanghai, China, shows microbial disequilibrium. Experimental dermatology, 24(5), pp. 398-400.

What is claimed is:

1. A method for restoring and/or maintaining a healthy skin microbiota, comprising at least one step of administering by topical route, to an individual, an effective amount of one or more live Lactobacillus species, wherein at least one of said Lactobacillus species is L. plantarum.

2. The method of claim 1, wherein L. plantarum is a L. plantarum strain having at least 97% sequence similarity with SEQ ID No 4 in its 16S rRNA gene.

3. The method of claim 1, wherein one or more of said Lactobacillus species further comprises L. pentosus.

4. The method of claim 3, wherein L. pentosus is a L. pentosus strain having at least 97% sequence similarity with SEQ ID No 1 in its 16S rRNA gene.

5. The method of claim 1, wherein one or more of said Lactobacillus species further comprises L. rhamnosus.

6. The method of claim 5, wherein L. rhamnosus is a L. rhamnosus strain having at least 97% sequence similarity with SEQ ID No 5 in its 16S rRNA gene.

7. The method for restoring and/or maintaining a healthy skin microbiota of claim 1, wherein said Lactobacillus species is a Lactobacillus strain selected from the list comprising L. plantarum YUN-V2.0 deposited under accession number LMG P-29456, L. pentosus YUN-V1.0 deposited under accession number LMG P-29455, and L. rhamnosus YUN-S1.0 deposited under accession number LMG P-29611.

8. The method of claim 1, wherein restoring a healthy skin microbiota comprises treating a skin disease relating to microbial dysbiosis.

9. The method of claim 8, wherein the microbial dysbiosis is caused by overgrowth of specific pathogenic micro-organisms.

10. The method of claim 8, wherein the skin disease is selected from the group consisting of Tinea pedis, Acnes vulgaris, atopic dermatitis, psoriasis, sensitive skin, folliculitis, impetigo, acne rosaceae, seborrheic eczema and dandruff.


SEQUENCE LISTING (7 sequences)

SEQ ID NO: 1 - RNA, 1406 nt, Lactobacillus pentosus
  16S rRNA sequence
cuuaggcggcugguuccuaaaagguuaccccaccgacuuuggguguuacaaacucucauggugugacgggcgguguguacaaggcccgggaacguauucaccgcggcaugcugauccgcgauuacuagcgauuccgacuucauguaggcgaguugcagccuacaauccgaacugagaauggcuuuaagagauuagcuuacucucgcgaguucgcaacucguuguaccauccauuguagcacguguguagcccaggucauaaggggcaugaugauuugacgucauccccaccuuccuccgguuugucaccggcagucucaccagagugcccaacuuaaugcuggcaacugauaauaaggguugcgcucguugcgggacuuaacccaacaucucacgacacgagcugacgacaaccaugcaccaccuguauccauguccccgaagggaacgucuaaucucuuagauuugcauaguaugucaagaccugguaagguucuucgcguagcuucgaauuaaaccacaugcuccaccgcuugugcgggcccccgucaauuccuuugaguuucagccuugcggccguacuccccaggcggaaugcuuaaugcguuagcugcagcacugaagggcggaaacccuccaacacuuagcauucaucguuuacgguauggacuaccaggguaucuaauccuguuugcuacccauacuuucgagccucagcgucaguuacagaccagacagccgccuucgccacugguguucuuccauauaucuacgcauuucaccgcuacacauggaguuccacuguccucuucugcacucaaguuucccaguuuccgaugcacuucuucgguugagccgaaggcuuucacaucagacuuaaaaaaccgccugcgcucgcuuuacgcccaauaaauccggacaacgcuugccaccuacguauuaccgcggcugcuggcacguaguuagccguggcuuucugguuaaauaccgucaauaccugaacaguuacucucagauauguucuucuuuaacaacagaguuuuacgagccgaaacccuucuucacucacgcggcguugcuccaucagacuuucguccauuguggaagauucccuacugcugccucccguaggaguuugggccgugucucagucccaauguggccgauuacccucucaggucggcuacguaucauugccauggugagccguuaccccaccaucuagcuaauacgccgcgggaccauccagaagugauagccgaagccaucuuucaaacucggaccaugcgguccaaguuguuaugcgguauuagcaucuguuuccagguguuaucccccgcuucugggcagguuucccacguguuacucaccaguucgccacucacucaaauguaaaucaugaugcaa

SEQ ID NO: 2 - DNA, 20 bp, Artificial Sequence
  Primer 8F
agagtttgatcctggctcag

SEQ ID NO: 3 - DNA, 20 bp, Artificial Sequence
  Primer 1525R
aaggaggtgatccagccgca

SEQ ID NO: 4 - RNA, 1425 nt, Lactobacillus plantarum
  16S rRNA
gguuccuaaaagguuaccccaccgacuuuggguguuacaaacucucauggugugacgggcgguguguacaaggcccgggaacguauucaccgcggcaugcugauccgcgauuacuagcgauuccgacuucauguaggcgaguugcagccuacaauccgaacugagaauggcuuuaagagauuagcuuacucucgcgaguucgcaacucguuguaccauccauuguagcacguguguagcccaggucauaaggggcaugaugauuugacgucauccccaccuuccuccgguuugucaccggcagucucaccagagugcccaacuuaaugcuggcaacugauaauaaggguugcgcucguugcgggacuuaacccaacaucucacgacacgagcugacgacaaccaugcaccaccuguauccauguccccgaagggaacgucuaaucucuuagauuugcauaguaugucaagaccugguaagguucuucgcguagcuucgaauuaaaccacaugcuccaccgcuugugcgggcccccgucaauuccuuugaguuucagccuugcggccguacuccccaggcggaaugcuuaaugcguuagcugcagcacugaagggcggaaacccuccaacacuuagcauucaucguuuacgguauggacuaccaggguaucuaauccuguuugcuacccauacuuucgagccucagcgucaguuacagaccagacagccgccuucgccacugguguucuuccauauaucuacgcauuucaccgcuacacauggaguuccacuguccucuucugcacucaaguuucccaguuuccgaugcacuucuucgguugagccgaaggcuuucacaucagacuuaaaaaaccgccugcgcucgcuuuacgcccaauaaauccggacaacgcuugccaccuacguauuaccgcggcugcuggcacguaguuagccguggcuuucugguuaaauaccgucaauaccugaacaguuacucucagauauguucuucuuuaacaacagaguuuuacgagccgaaacccuucuucacucacgcggcguugcuccaucagacuuucguccauuguggaagauucccuacugcugccucccguaggaguuugggccgugucucagucccaauguggccgauuacccucucaggucggcuacguaucauugccauggugagccguuaccccaccaucuagcuaauacgccgcgggaccauccaaaagugauagccgaagccaucuuucaagcucggaccaugcgguccaaguuguuaugcgguauuagcaucuguuuccagguguuaucccccgcuucugggcagguuucccacguguuacucaccaguucgccacucacucaaauguaaaucaugaugcaagcaccaaucaauaccagaguucguucgacu

SEQ ID NO: 5 - RNA, 1403 nt, Lactobacillus rhamnosus
  16S rRNA
gucgacgaguucugauuauugaaaggugcuugcaucuugauuuaauuuugaacgaguggcggacgggugaguaacacguggguaaccugcccuuaagugggggauaacauuuggaaacagaugcuaauaccgcauaaauccaagaaccgcaugguucuuggcugaaagauggcguaagcuaucgcuuuuggauggacccgcggcguauuagcuaguuggugagguaacggcucaccaaggcaaugauacguagccgaacugagagguugaucggccacauugggacugagacacggcccaaacuccuacgggaggcagcaguagggaaucuuccacaauggacgcaagucugauggagcaacgccgcgugagugaagaaggcuuucgggucguaaaacucuguuguuggagaagaauggucggcagaguaacuguugucggcgugacgguauccaaccagaaagccacggcuaacuacgugccagcagccgcgguaauacguagguggcaagcguuauccggauuuauugggcguaaagcgagcgcaggcgguuuuuuaagucugaugugaaagcccucggcuuaaccgaggaagugcaucggaaacugggaaacuugagugcagaagaggacaguggaacuccauguguagcggugaaaugcguagauauauggaagaacaccaguggcgaaggcggcugucuggucuguaacugacgcugaggcucgaaagcauggguagcgaacaggauuagauacccugguaguccaugccguaaacgaugaaugcuagguguuggaggguuuccgcccuucagugccgcagcuaacgcauuaagcauuccgccuggggaguacgaccgcaagguugaaacucaaaggaauugacgggggcccgcacaagcgguggagcaugugguuuaauucgaagcaacgcgaagaaccuuaccaggucuugacaucuuuugaucaccugagagaucagguuuccccuucgggggcaaaaugacagguggugcaugguugucgucagcucgugucgugagauguuggguuaagucccgcaacgagcgcaacccuuaugacuaguugccagcauuuaguugggcacucuaguaagacugccggugacaaaccggaggaagguggggaugacgucaaaucaucaugccccuuaugaccugggcuacacacgugcuacaauggaugguacaacgaguugcgagaccgcgaggucaagcuaaucucuuaaagccauucucaguucggacuguaggcugcaacucgccuacacgaagucggaaucgcuaguaaucgcggaucagcacgccgcggugaauacguucccgggccuuguacacaccgcccgucacaccaugagaguuuguaacacccgaagccgg

SEQ ID NO: 6 - DNA, 19 bp, Artificial Sequence
  Primer Sequence
tccgtaggtgaacctgcgg

SEQ ID NO: 7 - DNA, 20 bp, Artificial Sequence
  Primer Sequence
tcctccgcttattgatatgc